(19) United States
(12) Patent Application Publication
(10) Pub. No.: US 2023/0218536 A1
(43) Pub. Date: Jul. 13, 2023

(54) NANOPARTICLES CONTAINING COMPLEXES OF NUCLEIC ACIDS AND CATIONIC COPOLYMERS, PROCESS FOR PREPARING THEM AND THEIR USE FOR GENE TRANSFER IN CELLS

(71) Applicant: FRIEDRICH-SCHILLER-UNIVERSITÄT JENA (FSU), Jena (DE)

(72) Inventors: Jana Ines SOLOMUN, Jena (DE); Anja TRÄGER, Trebgast (DE)

(21) Appl. No.: 17/996,435

(22) PCT Filed: Apr. 14, 2021
(86) PCT No.: PCT/EP2021/000047

§ 371 Date: Oct. 18, 2022

(30) Foreign Application Priority Data
Apr. 18, 2020 (DE) 10 2020 002 360.6

(51) Int. Cl. A61K 9/51

(52) U.S. Cl. CPC A61K 9/5146; A61K 9/5192

(57) ABSTRACT

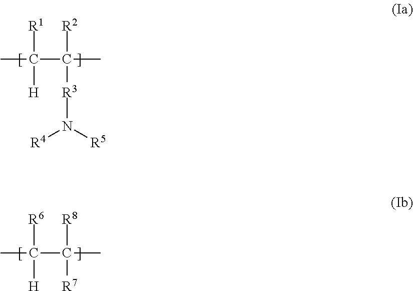

The invention relates to nanoparticles containing complexes constituted by nucleic acids and cationic copolymers containing the recurring structural units of formulae (Ia) and (Ib) wherein R¹ and R⁶ independently represent hydrogen, alkyl or —COOR⁹, R² and R⁷ independently represent hydrogen or alkyl, R³ is selected from the group consisting of —O—R¹⁰—, —COO—R¹⁰, —CONH—R¹⁰- or —R¹⁰—, R⁴ represents hydrogen, alkyl, cycloalkyl, aryl, aralkyl or alkylaryl, R⁵ represents hydrogen, alkyl, cycloalkyl, aryl, aralkyl, alkylaryl or —(alkylene-NH—)ₘalkyl, or R⁴ and R⁵ together with the nitrogen atom they have in common form a heterocyclic ring, R⁸ is selected from the group consisting of —O—R¹¹, —COO—R¹¹, —CONH—R¹¹ or —R¹¹, R⁹ and R¹¹ independently represent hydrogen or a monovalent organic residue, R¹⁰ represents a bivalent organic residue, and m is an integer from 1 to 5, with the proviso that the nanoparticles have a diameter (z-average) of less than or equal to 900 nm as determined by dynamic light scattering and that the molar ratio of nitrogen atoms in the copolymer to the phosphate groups in the nucleic acid ranges between 1 and 200. The nanoparticles according to the invention allow the transfer of nucleic acids into cells with great efficiency.

Nanoparticles comprising complexes of nucleic acids and cationic copolymers, methods for their preparation and their use for gene delivery into cells

The invention relates to the field of production and processing of nanoparticles comprising nucleic acid complexes with selected copolymers. These complexes can be advantageously used in the transfer of nucleic acids into cells.

EP 1 499 358 B1 describes combinations of nucleic acids with pH-sensitive polyacrylates. The production of nanoparticles is not disclosed. The copolymers containing (meth)acrylic acid described in this document are anionic depending on the pH. Cationic polymers are not disclosed.

WO 2018/130 247 A1 discloses nanoparticles with a carrier shell consisting of the components hydrophobic shell polymer, charged complexing polymer and hydrophilic active ingredient, including nucleic acids. The system described in this document consists of at least three components, with hydrophobicity and pH dependence divided between two different materials. The complexation polymer described in this document is a water-soluble linear polymer with amine functionalities, all of which are water-soluble at physiological pH values.

For some time the focus has been on cationic polymers as vehicles for gene delivery. Cationic groups such as primary, secondary and tertiary amines in the side chain, such as in 2-(dimethylamino)ethyl methacrylate (DMAEMA) or in the backbone, as found in linear polyethylenimine (IPEI), can bind and condense the genetic material through electrostatic interactions. This protects the genetic material from degradation by nucleases, for example, and at the same time allows it to be transported into cells. By introducing further hydrophobic units into cationic, water-soluble polymers, the in vitro efficiency of gene delivery can also be increased, inter alia, through improved interaction with cell membranes.

Copolymers comprising amino groups as well as hydrophobic structural units are known. One example is the polymer Eudragit® E. This copolymer is used to produce coatings for orally administrable medicinal products. The coating has the effect of, for example, masking the taste of the medicinal product. This is a copolymer derived from 2-(dimethylamino)ethyl methacrylate (DMAEMA), methyl methacrylate (MMA) and butyl methacrylate (BMA), which can also be called dimethylaminoethyl methacrylate copolymer.

It is further known to use dimethylaminoethyl methacrylate copolymer to increase the transfection efficiency of IPEI-DNA complexes (N. Kanthamneini, B. Yung, R. J. Lee, Anticancer Research 36: 81-86 (2016)). This paper reported that the combinations of DNA complexed with IPEI (=linear polyethylenimine) and dimethylaminoethyl methacrylate copolymer as an additive produced a synergistic effect on DNA gene expression compared to IPEI alone at low N/P ratios. Nanoparticles consisting of a combination of IPEI and (pEGFP)-DNA (=plasmid encoding an enhanced green fluorescent protein) were produced. Nanoparticles were also produced in which dimethylaminoethyl methacrylate copolymer was added. The proportion of dimethylaminoethyl methacrylate copolymer in the total mass of the nanoparticle was relatively low, less than 16% by weight. This document reported that it was not possible to prepare nanoparticles consisting only of dimethylaminoethyl methacrylate copolymer and (pEGFP) DNA. It was also stated in this document that the use of dimethylaminoethyl methacrylate copolymer alone as a cationic polymer, i.e. without IPEI, does not cause gene expression. Furthermore, this document referred to earlier documents describing studies on the use of nanoparticles of DNA and DMAEMA homopolymer in transfection experiments. However, the efficiency of these systems is inferior to that of IPEI.

In Med. Chem. Commun. 2015, 6, 691-701, R. Jain, P. Dandekar, B. Loretz, M. Koch and C. M. Lehr describe nanoparticles of dimethylaminoethyl methacrylate copolymer and siRNA. These particles were used to silence a therapeutically relevant gene (gene-silencing) in macrophages. The nanoparticles produced in this work have comparatively low mass fractions of bound siRNA. The described nanoparticles were produced in several steps using organic solvents. The genetic material was added to the finished nanoparticles and can thus only be bound to the surface of the nanoparticles, which is illustrated by a decrease in the zeta potential and an increase in the particle diameter (z-average) with increasing amounts of added genetic material. The siRNA-loaded cationic copolymer was used to promote the transfer of siRNA into the cytoplasm of macrophages. The nanoparticles were produced by introducing a solution of the cationic copolymer in the organic solvents acetone or ethyl acetate into an aqueous polyvinyl alcohol solution using a high-speed homogenizer. Stabilizers are needed to ensure sufficient stability of the nanoparticles. Instead of the protective colloid polyvinyl alcohol, surfactants were also used as stabilizers, e.g. vitamin E-TPGS or poloxamer 407. After evaporation of the solvent, a stabilized nanoparticle suspension was obtained, to which pDNA (pUC 18DNA) and then functional siRNA were added for stabilization. The nucleic acids can thus be bound to the surface of the nanoparticles (electrostatic interaction) and a core-shell structure of the nanoparticles is formed. In addition, these polymer-based particles contain protective colloids or surfactants for stabilization. However, the use of such stabilizers must be viewed critically, as the particle properties are influenced by them, for example the surface charge (or zeta potential) of the particles. For example, polyvinyl alcohol as well as other surfactants have a cell-damaging effect at high concentrations and consequently entail a situation whereby produced particles must subsequently be purified. Gene expression is not described or shown in this work, although pDNA was also used.

In Pharmaceutical Research, vol. 26, No. 1, January 2009, 72-81, A. Basarkar and J. Singh describe nanoparticles made from a combination of poly(lactide-co-glycolide) and dimethylaminoethyl methacrylate copolymer. These particles were loaded with a plasmid encoding a mouse interleukin-10 gene. The nanoparticles were produced by introducing a solution of the polymers in the organic solvent dichloromethane into an aqueous solution buffered with phosphate. The proportion of cationic copolymers was up to 50% by weight of the total amount of polymers. A w/o emulsion was obtained by sonication with ultrasound. The cationic surfactant cetyltrimethylammonium bromide (CTAB) was added to this and sonicated again, resulting in a w/o/w emulsion. The organic solvent was evaporated, the nanoparticles were separated by centrifugation and excess surfactant was removed. The nanoparticles were then freeze-dried. The plasmid was loaded by suspending the finished nanoparticles in a plasmid solution. The nucleic acids are thereby bound to the surface of the nanoparticles by electrostatic interactions and a core-shell structure of the nanoparticles is formed. In addition, these polymer-based particles contain cationic surfactants for stabilization, which can induce a positive charge on the surface of the particles and thereby contribute to the binding of the genetic material.

In J. Mater. Chem. B 2014, 2, 7123-7131, G. Doerdelmann, D. Kozlova and M. Epple describe a pH-sensitive poly(methyl methacrylate) copolymer for use as an effective agent for drug and gene delivery across a cell membrane. It describes a system of Ca-phosphate/dimethylaminoethyl methacrylate copolymer nanoparticles with diameters of less than 200 nm in the form of a water-in-oil-in-water emulsion. These particles were produced by making Ca-phosphate nanoparticles loaded with siRNA by mixing aqueous solutions of Ca-lactate and of ammonium hydrogen phosphate and adding them to an aqueous solution of anti-EGFP siRNA with vigorous stirring. This formed a dispersion of nanoparticles from a core of Ca-phosphate coated by the anti-EGFP-siRNA. These nanoparticles were then encapsulated in the dimethylaminoethyl methacrylate copolymer by adding the suspension to a solution of the copolymer in dichloromethane. After addition of an aqueous solution of calf serum albumin (BSA), this was sonicated with ultrasound to form a primary W/O emulsion. This was poured into water to which polyvinyl alcohol was added as a dispersant and again sonicated with ultrasound. After 3 hours of vigorous stirring, the dichloromethane had evaporated and nanoparticles had formed with a core-shell structure of Ca-Phosphate/anti-EGFP-siRNA surrounded by a shell of dimethylaminoethyl methacrylate copolymer.

Known particles comprising combinations of dimethylaminoethyl methacrylate copolymer with nucleic acids thus have a core-shell structure, wherein the nucleic acids are on the surface of a polymer core or a core of inorganic material, or the nucleic acids are surrounded by dimethylaminoethyl methacrylate copolymer, i.e. not bound, or dimethylaminoethyl methacrylate copolymer is combined with an excess of IPEI. Often these known nanoparticles also contain surfactants or protective colloids as stabilizers, which can have an influence on the charge and interaction with the genetic material and cells.

It has now been surprisingly found that nanoparticles can be produced which contain complexes of nucleic acids and selected cationic copolymers, wherein the nucleic acid is better complexed and condensed by the cationic copolymer than in complexes produced by known methods. This manifests itself, inter alia, in a smaller particle diameter of the nanoparticles produced according to the invention compared to conventionally produced nanoparticles, in particular with a high proportion of genetic material.

One object of the present invention was to provide nanoparticles which have a compact and simple structure and a high content of nucleic acid-copolymer complexes and which are ideally suited for gene delivery of nucleic acids.

Another object of the present invention was to provide nucleic acid copolymer nanoparticles which are preferably free of any stabilizers.

A further object of the present invention was to provide a nanoparticle production method that does not require organic solvents, can be performed simply, quickly and reproducibly and results in nucleic acid-copolymer complexes with high gene delivery efficiency.

The present invention relates to nanoparticles comprising complexes formed from nucleic acids and cationic copolymers containing the recurring structural units of the formulae (Ia) and (Ib)

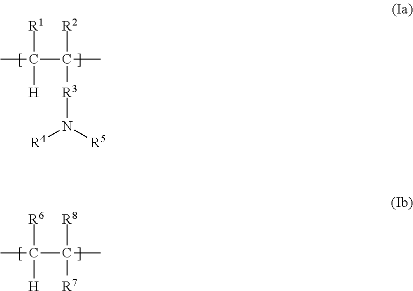

wherein

R¹ and R⁶ are, independent of each other, hydrogen, alkyl or —COOR⁹,

R² and R⁷ are, independent of each other, hydrogen or alkyl,

R³ is selected from the group consisting of —O—R¹⁰—, —COO—R¹⁰—, —CONH—R¹⁰— or —R¹⁰—

R⁴ is hydrogen, alkyl, cycloalkyl, aryl, aralkyl or alkylaryl,

R⁵ is hydrogen, alkyl, cycloalkyl, aryl, aralkyl, alkylaryl or —(alkylene-NH—)ₘ-alkyl, or

R⁴ and R⁵ together with the nitrogen atom they have in common form a heterocyclic ring,

R⁸ is selected from the group consisting of —O—R¹¹, —COO—R¹¹, —CONH—R¹¹ or —R¹¹

R⁹ and R¹¹ are, independent of each other, hydrogen or a monovalent organic radical,

R¹⁰ is a bivalent organic radical, and

m is an integer from 1 to 5, with the requirement that

the nanoparticles have a diameter (z-average), determined by dynamic light scattering, of less than or equal to 900 nm, and that the molar ratio of nitrogen atoms in the copolymer to the phosphate groups in the nucleic acid is between 1 and 200.

In the nanoparticles according to the invention, DNA and/or RNA and modifications thereof can be used as nucleic acids.

Any DNA types can be used. Examples are A-DNA, B-DNA, Z-DNA, mtDNA, antisense DNA, bacterial DNA and viral DNA.

Any RNA types can also be used. Examples include hnRNA, mRNA, tRNA, rRNA, mtRNA, snRNA, snoRNA, scRNA, siRNA, miRNA, antisense RNA, bacterial RNA and viral RNA.

Combinations of DNA and RNA can also be used in the complexes according to the invention.

The cationic copolymers used in the complexes according to the invention are copolymers which contain at least one recurring structural unit of the formula (Ia), which is derived from an ethylenically unsaturated monomer containing an amino group, and which contain at least one further recurring structural unit of the formula (Ib), preferably at least two different structural units of the formula (Ib), which are derived from ethylenically unsaturated monomers comprising a hydrocarbon radical.

The cationic copolymers used in the complexes according to the invention may contain one or more different recurring structural units of formula (Ia). Preferably, these copolymers contain only one type of the recurring structural units of formula (Ia).

The cationic copolymers used in the complexes according to the invention may contain one or more different recurring structural units of formula (Ib). Preferably, these copolymers contain only one or two different types of the recurring structural units of formula (Ib) in addition to the recurring structural units of formula (Ia).

In addition to the recurring structural units of formulae (Ia) and (Ib), the cationic copolymers used in the complexes according to the invention may contain further recurring structural units of formula (Ic)

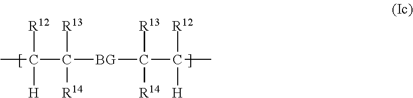

wherein

R¹², R¹³ and R¹⁴ are, independent of each other, hydrogen or alkyl, preferably hydrogen or C₁-C₆ alkyl, particularly preferably hydrogen or methyl, and BG means a bivalent organic bridging group with ether, ester, amide, sulfide, phosphate or disulfide groups.

The presence of structural units of formula (Ic) in the copolymer used according to the invention confers improved biodegradability upon it.

Examples of polymers from which recurring structural units of formula (Ic) are derived are polyesters, polyamides, polyethers, phosphates, sulfides or disulfides, each having two end groups with ethylenically unsaturated groups, such as vinyl or allyl groups.

The radicals R¹, R², R⁴-R⁷ and R¹²-R¹⁴ can mean alkyl. These are usually alkyl groups with one to six carbon atoms, which can be straight-chain or branched. Methyl and ethyl are preferred, particularly methyl.

The radicals R⁴ and R⁵ can mean cycloalkyl. These are usually cycloalkyl groups with five to six ring carbon atoms. Cyclohexyl is particularly preferred.

The radicals R⁴ and R⁵ can mean aryl. These are usually aromatic hydrocarbon radicals with five to ten ring carbon atoms. Phenyl is preferred.

The radicals R⁴ and R⁵ can mean alkylaryl. These are usually aryl groups substituted with one or two alkyl groups. Tolyl is preferred.

The radicals R⁴ and R⁵ can mean aralkyl. These are usually aryl groups which are connected to the rest of the molecule via an alkylene group. Benzyl is preferred.

R⁵ can also be a radical of the formula —(alkylene-NH—)ₘ-alkyl. The alkylene radicals thereby usually have two to four carbon atoms and can be branched or preferably straight-chain. The alkyl group usually has one to four carbon atoms and is preferably ethyl or methyl, particularly methyl. The number of recurring units, characterized by the index m, is 1 to 5, preferably 1 to 3.

R⁴ and R⁵ can also form a heterocyclic ring together with the nitrogen atom they have in common. These are usually rings with a total of five to six ring atoms, of which one or two ring atoms are heteroatoms and the rest of the ring atoms are carbon atoms. One of the ring heteroatoms is a nitrogen atom. An additional ring heteroatom, if present, is nitrogen, oxygen or sulfur.

R⁹ and R¹¹ can, independent of each other, be monovalent organic radicals. These are organic radicals with a covalent bond that establishes the connection to the rest of the molecule. The monovalent organic radicals are usually alkyl, cycloalkyl, aryl, alkylaryl or aralkyl.

R¹⁰ is a bivalent organic radical. These are organic radicals with two covalent bonds that establish the connection to the rest of the molecule. Bivalent organic radicals are usually alkylene, cycloalkylene, arylene, alkylarylene or aralkylene.

Preferably, copolymers are used in which R¹ and R⁶, independent of each other, denote hydrogen or C₁-C₆ alkyl, particularly hydrogen or methyl, and very particularly preferably hydrogen.

Preferably, copolymers are used in which R² and R⁷, independent of each other, denote hydrogen or C₁-C₆ alkyl, particularly hydrogen or C₁-C₆ alkyl, particularly preferably hydrogen or methyl and particularly preferably methyl.

Preferably, copolymers are used in which R³ is a bivalent radical of the formulae —O—R¹⁰—, —COO—R¹⁰—, —CONH—R¹⁰— or —R¹⁰—, and R¹⁰ is selected from the group consisting of C₂-C₁₂ alkylene, C₅-C₇ cycloalkylene and C₆-C₁₀ arylene, particularly C₂-C₆ alkylene, and very particularly preferably ethylene.

Preferably, copolymers are used in which R⁴ and R⁵, independent of each other, denote hydrogen or C₁-C₆ alkyl, and particularly hydrogen and methyl and very particularly preferably methyl.

Preferably, copolymers are used in which R⁸ is a monovalent radical of the formulae —O—R¹¹, —COO—R¹¹, —CONH—R¹¹ or —R¹¹, and R¹¹ is alkyl, alkenyl, cycloalkyl, aryl, aralkyl or alkylaryl, particularly C₁-C₆ alkyl, vinyl, allyl, phenyl, benzyl or C₁-C₆ alkylphenyl and very particularly preferably C₁-C₆ alkyl.

Particularly preferably, copolymers are used in which R⁸ represents —COO—R¹¹ or —CONH—R¹¹, and R¹¹ denotes C₁-C₆ alkyl, phenyl, benzyl or C₁-C₆ alkylphenyl, very particularly preferably C₁-C₆ alkyl and extremely preferably methyl, ethyl, propyl and/or butyl.

Preferably, copolymers are used in which R⁹ and R¹¹, independent of each other, denote alkyl, cycloalkyl, aryl, aralkyl or alkylaryl, particularly C₁-C₆ alkyl, phenyl, benzyl or C₁-C₆ alkylphenyl and very particularly preferably C₁-C₆ alkyl.

Preferably, copolymers are used in which R¹⁰ denotes C₂-C₁₂ alkylene, C₅-C₇ cycloalkylene and C₆-C₁₀ arylene, particularly C₂-C₆ alkylene, and very particularly preferably ethylene.

Particularly preferably, copolymers are used containing a recurring structural unit of the formula (Ia) and two different recurring structural units of the formula (Ib), in which R¹ and R⁶ denote hydrogen, R² and R⁷, independent of each other, are hydrogen or methyl, particularly methyl, R³ is —COO—R¹⁰—, R¹⁰ denotes ethylene, R⁴ and R⁵, independent of each other, are C₁-C₆ alkyl, particularly methyl, and R⁸ is —COO—R¹¹, wherein, in a recurring structural unit of the formula (Ib) R¹¹ is C₁-C₃ alkyl, particularly methyl, and, in another recurring structural unit of the formula (Ib) R¹¹ is C₄-C₆ alkyl, particularly n-butyl.

It is assumed that the compact nanoparticles according to the invention contain nucleic acid-copolymer complexes which are distributed over the entire volume of the nanoparticle. In contrast to previously known nanoparticles with a pronounced core-shell structure and with a concentration of the nucleic acid-polymer complexes in the outer shell, in the nanoparticles according to the invention nucleic acid-copolymer complexes are found both in the interior and in the exterior regions of the nanoparticles. Such nanoparticles are also referred to hereinafter as “polyplexes”.

The nanoparticles according to the invention are further characterized by a high content of nucleic acid. The proportion by weight of cationic copolymer containing the recurring structural units of the formula (Ia) and (Ib) in the nanoparticles according to the invention is typically 15 to 99%, and preferably 20 to 90% and particularly 30 to 80%, based on the total mass of the nanoparticles.

The nanoparticles according to the invention can be characterized by their particle diameter. Typical particle diameters (for example z-average) are in the range of less than or equal to 900 nm, preferably less than or equal to 500 nm, particularly preferably between 30 and 500 nm, very particularly preferably between 40 and 250 nm and especially between 50 and 200 nm. The particle diameters are determined for the purposes of the present description by dynamic light scattering (DLS) using a Malvern Zetasizer Nano ZS (Malvern Instruments, Worcestershire, United Kingdom). Cumulant analysis of the correlation function (ISO13321, ISO22412) was used to determine the intensity-weighted mean diameter (e.g. z-average). For sizing, a refractive index of 1.33 was assumed for ultra pure water and 1.59 for the copolymer.

Particle diameters can alternatively be determined by other methods, for example by nanosize tracking analysis (NTA), or by electron microscopy, e.g. using a transmission electron microscope or a scanning electron microscope.

Particle diameters (z-average) of preferred nanoparticles according to the invention range between 50 and 200 nm, determined by dynamic light scattering (DLS).

The cationic copolymers used according to the invention and the nanoparticles according to the invention can be further characterized by their polydispersity index (or PDI). The polydispersity index Ð or PDI_{MW} of molecular weights is a measure of the breadth of the molecular weight distribution of a polymer. The polydispersity index Ð or PDI_{MW} is calculated from the ratio of the weight average to the number average of the molecular weight distribution. The greater Ð, the broader the molecular weight distribution. In the case of very narrow molecular weight distributions, the value of PDI_{MW} tends towards 1. In the case of broader molecular weight distributions, the value of PDI_{MW} is significantly greater than 1.

The polydispersity index of particle size distribution PDI_{TG}, on the other hand, indicates the breadth of particle size distribution for particles. Values between 0 (monodisperse) and 1 (polydisperse) can thereby be assumed. The PDI_{TG} value is determined for the purpose of the present description by dynamic light scattering (DLS) using a Malvern Zetasizer Nano ZS (Malvern Instruments, Worcestershire, United Kingdom). PDI_{TG} was determined by means of cumulant analysis of the correlation function.

The PDI_{TG} value of the particle size distribution of the nanoparticles according to the invention typically ranges between 0.05 and 0.4, preferably between 0.1 and 0.4, and particularly preferably between 0.1 and 0.3.

The polydispersity index Ð of the molecular weight distribution of the cationic copolymers used according to the invention typically ranges between 1.0 and 5.0, preferably between 1.01 and 2.6.

The nanoparticles according to the invention can also be characterized by their transfection efficiency for DNA. For this purpose, nanoparticles comprising a selected protein-coding DNA, for example eGFP-coding DNA, are brought into contact with cells for a predetermined time, for example 1 hour, and incubated. The cells are then incubated in growth medium without nanoparticles for 23 hours, after which it is determined how many of the cells express the selected protein. The proportion of expressing cells, specified as a percentage, is used to represent the transfection efficiency for DNA.

Preferred nanoparticles according to the invention exhibit a transfection efficiency for DNA of 15 to 50% (viable fluorescent cells), particularly of 20 to 45%, after 1 hour of incubation.

The nanoparticles according to the invention can be further characterized by their N/P ratio. This is the molar ratio of nitrogen atoms in the copolymer to phosphate groups in the nucleic acid.

The N/P ratio in the nanoparticles according to the invention can vary in wide ranges. Typically, the N/P ratio in the nanoparticles according to the invention is between 1 and 200, preferably between 1 and 100, especially between 1 and 50, particularly preferably between 1 and 30, very particularly preferably between 2.5 and 100, extremely preferably between 5 and 50, and most preferably between 5 and 30.

Preferred nanoparticles according to the invention have diameters (z-average) determined by DLS between 40 and 250 nm, particularly between 50 and 200 nm, and a polydispersity index of particle diameters between 0.1 and 0.3.

Very particularly preferred nanoparticles according to the invention have diameters (z-average) determined by DLS between 40 and 250 nm, particularly between 50 and 200 nm, and a polydispersity index of particle diameters between 0.1 and 0.3 and an N/P ratio between 10 and 30.

The molar proportion of recurring structural units of formula (Ia) in the cationic copolymers used according to the invention is usually between 10 and 75%, preferably between 15 and 65% and very preferably between 20 and 55%, based on the total cationic copolymer.

The molar proportion of recurring structural units of formula (Ib) in the cationic copolymers used according to the invention is usually between 90 and 25%, preferably between 85 and 45% and very preferably between 80 and 45%, based on the total cationic copolymer.

The molar proportion of recurring structural units of formula (Ic) in the cationic copolymers used according to the invention is usually between 0 and 25%, preferably between 1 and 10% and very preferably between 5 and 10%, based on the total cationic copolymer.

Preferably, nanoparticles according to the invention are used which do not contain any excipients or additives, in particular no protective colloids and/or surfactants.

In the event that the nanoparticles according to the invention contain additional polymers or additional complexes of nucleic acids with additional polymers, e.g. complexes of nucleic acids with IPEI, in addition to the nucleic acid-copolymer complexes described above, these further components are present only in small amounts, for example their proportion by weight is 15% or less, particularly less than 5%.

Particularly preferably, the nanoparticles according to the invention do not contain any further complexes of nucleic acids with other polymers in addition to the nucleic acid-copolymer complexes described above.

The terms for “particles” [German: Teilchen/Partikel: particles] are used synonymously in the context of the present description.

In the context of the present description, “nanoparticles” are understood to be particles whose diameter (z-average) is less than or equal to 900 nm and which may be composed of cationic copolymers as well as complexes thereof with nucleic acids or only of such complexes. They are generally characterized by a very high surface-to-volume ratio and thus offer very high chemical reactivity. Nanoparticles may only consist of the aforementioned cationic copolymers and complexes or only of the complexes, or may also contain other components in addition to the copolymers and complexes, such as active agents or excipients or additives.

In the context of the present description, “copolymers” are understood to be the above-mentioned organic compounds which are characterized by the repetition of at least two different specific units (monomer units or repetition units). Copolymers are produced by the chemical reaction of monomers with the formation of covalent bonds (polymerization) and form what is called the polymer backbone by linking the polymerized units. This can have side chains on which functional groups can be located. Some of the copolymers have hydrophobic properties and, depending on the concentration, can form nanoscale structures (e.g. nanoparticles, micelles, vesicles) in an aqueous environment. The copolymers consist of at least two, preferably three different monomer units, which can be arranged statistically, as a gradient or alternately.

In the context of the present description, “surfactants” are understood to be non-polymeric substances or mixtures of substances which have water-soluble and water-insoluble properties and which serve to stabilize particles during production and storage in aqueous media. They are usually added to the dispersing medium, e.g. the aqueous phase, during the production of the particles, but can also be added after their production to stabilize the obtained dispersion. For example, cationic surfactants could be added to the surface of the nanoparticles to shift their surface charge and thus enable nucleic acids to bind on the surface, for example by coating with CTAB (cetyltrimethylammonium bromide).

In the context of the present description, “protective colloids” are understood to be water-soluble or water-dispersible polymers or polymer mixtures which serve to stabilize particles during production and storage in aqueous media. They are usually added to the dispersing medium, e.g. the aqueous phase, during the production of the particles, but can also be added after their production to stabilize the obtained dispersion.

In the context of the present description, “water-soluble compounds” or “water-soluble polymers” are understood to be compounds or polymers that dissolve to at least 1 g/L water at 25° C. and at neutral pH values.

In the context of the present description, “excipients and additives” are understood to be substances that are added to a formulation to give it certain additional properties and/or to facilitate its processing. Examples of excipients and additives are tracers, contrast agents, carriers, fillers, pigments, dyes, perfumes, slip agents, UV stabilizers, antioxidants or surfactants. In particular, “excipients and additives” are understood to be any pharmacologically acceptable and therapeutically useful substance which is not a pharmaceutically active agent but which can be formulated together with a nucleic acid in a nucleic acid-copolymer complex in order to influence, in particular improve, qualitative properties of the nanoparticle. Preferably, the excipients and/or additives have no effect or no significant effect or at least no undesirable effect with regard to the intended procedure.

In the context of the present description, “gene delivery” is understood to be the introduction of nucleic acids into cells and their functional release in the cells.

The nanoparticles according to the invention may be present in solid form as a powder or they may form a dispersion and be dispersed in aqueous solvents, the particles being present in the dispersing medium in solid form.

In a preferred embodiment, the nanoparticles according to the invention form a disperse phase in water or in an aqueous buffer solution.

The solubility of the copolymers used according to the invention can be influenced by co-polymerization with suitable monomers and by functionalization. Such techniques are known to the person skilled in the art.

The proportion of nanoparticles according to the invention in a dispersion can cover a broad range. Typically, the proportion by weight of nanoparticles in the dispersion is between 0.01 and 20%, preferably between 0.05 and 5%.

The organic copolymers used according to the invention can cover a broad range of molar masses. Typical molar masses (Mₙ) range from 2,000 to 500,000 g/mol, in particular from 5,000 to 50,000 g/mol. These molar masses can be determined by ¹ H—NMR spectroscopy of the dissolved copolymer. In particular, an analytical ultracentrifuge or chromatographic methods, such as size exclusion chromatography, can be used to determine the molar masses.

Preferred organic copolymers have an average molar mass (number average) in the range of 5,000 to 40,000 g/mol, determined by ¹H—NMR spectroscopy or by using an analytical ultracentrifuge.

The cationic copolymers used according to the invention can be produced using the usual polymerization methods. Examples are polymerization in substance, polymerization in solution, or emulsion or suspension polymerization. These methods are known to the person skilled in the art.

The nanoparticles according to the invention can be produced by nanoprecipitation. For this purpose, the cationic copolymers used according to the invention, which are hydrophilic depending on the pH value due to the presence of polar groups, are dissolved in water or in an aqueous buffer solution. The pH of the aqueous solution is adjusted to a value between 3 and 6.5, e.g. by using an acetate buffer or another suitable buffer such as, for example, citrate buffer, lactate buffer, phosphate buffer and phosphate-citrate buffer. In addition, the nucleic acids are dissolved in water, whereby the pH of the aqueous nucleic acid solution is preferably adjusted to a value between 6.5 and 8.5, particularly preferably to a value between 6.8 and 7.5. A buffer solution containing HEPES, TRIS, BIS-TRIS-propane or only salts is particularly suitable for this purpose. Both solutions are combined, whereby the amounts of nucleic acids and cationic copolymer are chosen in such a way that a desired N/P ratio is obtained. After mixing the two solutions, the mixture is agitated, for example for a short time, such as between 2 and 20 seconds. This may be done by stirring and/or by vortexing. Preferably, the resulting nanoparticles are left to stand for some time, for example between 5 and 20 minutes, before further use, to allow binding between the polymer and the nucleic acids (hereinafter referred to as “incubation”). The nanoparticles according to the invention are precipitated in the dispersing medium in finely dispersed form.

In addition to the cationic copolymer and the nucleic acid, one or more excipients and additives may be present during their nanoprecipitation in the dispersing medium. Alternatively, these excipients and additives may be added after the nucleic acid-copolymer complex has been dispersed in the aqueous phase.

Water is used as the dispersing medium. Buffer substances, salts, sugars or acids and bases can be added to this to adjust the desired pH value or osmolarity.

The invention also relates to a method for the production of nanoparticles, which comprises the following measures:

i) preparing an aqueous solution of a cationic copolymer containing the recurring structural units of formulae (Ia) and (Ib) described above with a pH between 3 and 6.5,
ii) preparing an aqueous solution of a nucleic acid,
iii) mixing both solutions prepared in steps i) and ii) in a chosen quantity ratio of nucleic acid and copolymer, such that a desired molar N/P ratio of nitrogen atoms in the copolymer to the phosphate groups in the nucleic acid is obtained, i.e. an N/P ratio between 1 and 200,
iv) agitating the mixture from step iii), and
v) subsequently incubating the resulting mixture, as appropriate.

The aqueous solution of the cationic copolymer for step i) of the method according to the invention preferably contains a buffer, particularly an acetate buffer, citrate buffer, lactate buffer, phosphate buffer, phosphate-citrate buffer or mixtures thereof.

The aqueous solution of the nucleic acid for step ii) of the method according to the invention preferably has a pH between 6.5 and 8.5, particularly between 6.8 and 7.5.

The aqueous solution of the nucleic acid for step ii) of the method according to the invention preferably contains a buffer, particularly a HBG, HEPES, BIS-TRIS propane or TRIS buffer.

The agitation in step iv) of the method according to the invention is preferably carried out by stirring or vortexing. The processing duration in this step is usually between 1 and 60 seconds, particularly between 2 and 20 seconds.

The incubation in step v) of the method according to the invention is usually carried out by simply leaving the obtained mixture to stand, for example for a period of 5 to 60 minutes, preferably from 5 to 20 minutes. The mixture may also be incubated in an oven, for example at temperatures between 30 and 80° C.

The separation of the nanoparticles from the aqueous phase can be achieved in various ways. Examples are centrifugation, ultrafiltration or dialysis. However, the dispersion of nanoparticles can also preferably be used directly after production without further processing.

Purification by means of filtration can separate particles, such as aggregates, but also excess excipients or impurities from the dispersion. The particle concentration can thereby change.

Purification by dialysis can separate dissolved molecules from the dispersion. This method is largely independent of the particle size with regard to the dispersed particles.

Purification by centrifugation can also separate dissolved molecules from the dispersion. However, this method also reduces the concentration of the dispersed particles. Furthermore, only dispersions with nanoparticles of larger diameter, e.g. of more than 150 nm, can be treated and the particles may be affected. Furthermore, redispersing the particles obtained in this way can cause difficulties.

The nanoparticles according to the invention are excellently suited for gene delivery in cells, i.e. for the introduction of nucleic acids into cells. For this purpose, the nanoparticles comprising nucleic acids are added to individual cells, tissues or a cell culture and taken up by the cells by endocytosis. Surprisingly, it has been shown that high contents of nucleic acids can be transferred into cells by means of the nanoparticles according to the invention. Nanoparticles comprising a comparatively low cationic copolymer content can be used for this.

The invention therefore also relates to a method for gene delivery into cells, which comprises the following steps:
A) Bringing cells into contact with an aqueous suspension comprising the nanoparticles described above and
B) Subsequent incubation.

Preferably, the invention relates to a method for gene delivery into cells comprising the following steps:
C) Provision of a cell culture in a bioreactor or incubator,
D) Addition of an aqueous suspension comprising the nanoparticles described above,
E) Distribution of the aqueous suspension in the cell culture, and
F) Subsequent incubation.

The gene delivery method according to the invention can be carried out using various cells, for example by using single cells, tissues or cell cultures.

Thus, the nanoparticles according to the invention can be combined with prokaryotic cells, with tissues from eukaryotic cells or with cell cultures. These can be plant cells or preferably animal cells, including human cells.

The application of the nanoparticles according to the invention can take place in vivo, for example under the skin or in the muscle, or the application can also take place ex vivo, for example with immune cells, as in CAR-T therapy. It can also be an RNA vaccination or an immunization.

In the context of the present description, “cells” are understood to be the smallest living units of organisms. They may be cells of unicellular or multicellular organisms, which may originate from prokaryotes or eukaryotes. Cells may be microorganisms or single cells. Cells may be of prokaryotic, plant or animal origin or may also originate from fungi. Preferably, eukaryotic cells are used, in particular eukaryotic cells which were originally isolated from tissue and can be cultivated permanently, i.e. that are immortalized.

In the context of the present description, “tissues” are understood to be collections of differentiated cells including their extracellular matrix.

In the context of the present description, “cell cultures” refers to combinations of cells and cell culture medium, whereby the cells are cultivated in the cell culture medium outside the organism. For this purpose, cell lines are used, i.e. cells of a tissue type that can divide in the course of cultivation. Both immortalized (immortal) cell lines and primary cells (primary culture) can be cultivated. Primary culture is usually understood to mean a non-immortalized cell culture obtained directly from a tissue.

In the context of the present description, “cell culture medium” or “nutrient medium” are understood to mean aqueous systems that serve as a platform for the cultivation of cells. These systems contain all the substances required for the growth and viability of the cells.

The cell culture medium may contain serum in addition to the cells and the required nutrients. Preferably, the cell culture medium contains sera or proteins and growth factors.

In the context of this description, “serum” is understood to mean blood serum or immune serum. Blood serum is thereby understood to be the liquid portion of the blood that is obtained as supernatant when a blood sample is centrifuged. This supernatant contains all substances naturally dissolved in the blood fluid except for the coagulation factors consumed by coagulation. The blood serum thus corresponds to the blood plasma minus the coagulation factors. Immune serum is understood to be a purification of specific antibodies obtained from the blood serum of immunized mammals.

Sera in the context of this description usually mean sera from vertebrates, and in particular sera from calf, cow, bull, horse or human.

The cell cultures used according to the invention can be produced and cultivated according to standard methods.

For example, primary cultures can be created from various tissues, e.g. from tissues of individual organs such as skin, heart, kidney or liver, or from tumor tissue. The tissue cells can be isolated by methods as known per se, e.g. by treatment with a protease, which degrades the proteins that maintain the cell bond. It may also be appropriate to specifically stimulate some cell types to divide by adding growth factors or, in the case of poorly growing cell types, to use feeder cells, basement membrane-like matrices or recombinant extracellular matrix components. The cells used according to the invention can also be genetically modified by introducing a plasmid as a vector.

The cells used according to the invention may have a limited lifespan or they may be immortal cell lines with the ability to divide infinitely. These may have been generated by random mutation, e.g. in tumor cells, or by targeted modification, for example by the artificial expression of the telomerase gene.

The cells used according to the invention can be adherent cells (growing on surfaces), such as fibroblasts, endothelial cells or cartilage cells, or they can be suspension cells that grow freely floating in the nutrient medium, such as lymphocytes.

Culture conditions and cell culture media are selected depending on the individual cells being cultured. The different cell types thereby prefer different nutrient media, which are composed specifically. For example, different pH values are established and the individual nutrient media can contain various amino acids and/or other nutrients in various concentrations.

The cells transfected according to the invention can be used in various fields, for example biotechnology, research or medicine. This may involve the production of (recombinant) proteins, virus and/or virus particle production, investigation of metabolism, division and other cellular processes. Furthermore, the cells transfected according to the invention can be used as test systems, for example in the investigation of the effect of substances on cell properties, such as signal transduction or toxicity. Further cells preferably used for the production of the cells transfected according to the invention are stem cells. These are known to be body cells that can diversify into various cell types or tissues.

The invention also relates to the use of the nanoparticles described above for gene delivery into cells, i.e. for introducing nucleic acids into cells.

The following examples illustrate the invention without limiting it.

The synthesis of polymers by RAFT polymerization, comparable to the commercially available product EUDRAGIT® E100, is described below. The suitability of the polymers for nucleic acid binding was investigated and a novel method of nucleic acid encapsulation and formulation is described.

Abbreviations

The following abbreviations are used in the examples:

CPAETC: 4-cyano-4-(phenylcarbonothioylthio)pentanoic acid

nBMA: n-butyl methacrylate

MMA: methyl methacrylate

DMAEMA: 2-(N,N-dimethylamino)ethyl methacrylate

ACVA: 4,4′-azobis-(4-cyanovaleric acid)

DMAc-SEC: Size exclusion chromatography with dimethylacetamide+0.21% LiCl as eluent

CTA: Chain Transfer Agent

pDNA: mEGFP-N1 plasmid (coding for EGFP); pKMyc plasmid (control plasmid)

HEPES: 2-(4-(2-hydroxyethyl)-1-piperazinyl)-ethanesulfonic acid

HBG: 5% glucose solution buffered with HEPES.

DMEM: Dulbecco's modified Eagle Medium

HBSS: Salt solution according to Hanks

FBS: Foetal calf serum

EGFP: enhanced green fluorescent protein

IPEI: linear polyethylenimine

PDMAEMA: homopolymeric 2-(N, N-dimethylamino)ethyl methacrylate

PBMD: nBMA-st-MMA-st-DMAEMA copolymer (st=statistically distributed)

PDI: Polydispersity index determined by DLS using a Malvern Zetasizer Nano ZS (Malvern Instruments, Worcestershire, United Kingdom) applying cumulant analysis of the correlation function (ISO13321, ISO22412).

RFI: relative fluorescence intensity

CTRL: Control in the form of cells treated only with HBG buffer and not with copolymer.

E100: EUDRAGIT® E100 (in powder form)

Materials and Methods

The following materials were used in the subsequent experiments:

E100: EUDRAGIT® E100 in powder form

pDNA (eGFP, pkmyc): mEGFP-N1 plasmid (coding for EGFP), pKMyc plasmid (control plasmid, not coding for a fluorescent protein)

Cells: Human embryonic kidney (HEK) cells, in particular the HEK293T cell line.

Agarose: Agarose-HR plus

Molecular weight calculation of the polymers produced during RAFT polymerization

The monomer conversion (p) was calculated from ¹H—NMR data by comparing the integrals of the vinyl bands (5.5-6.3 ppm) with an external reference (1,3,5-trioxane, 5.14 ppm) before (t=0) and after (t=final) polymerization. The theoretical number average molar mass (Mₙ, ₜₕ) was then calculated using the following equation:

Mₙ, ₜₕ=(M))₀*p*M_{M})/[CTA]₀)+M_{CTA}

wherein [M]₀ and [CTA]₀ are the initial concentrations of monomer and CTA, respectively, M_{M} and M_{CTA} are the molecular weight of monomer and CTA, respectively, and p is the conversion of monomer to CTA.

Production and Characterization of Polymers and of Nanoparticulate Polymer Particles or Nanoparticulate DNA-Polymer Complexes

### EXAMPLE 1A

### Synthesis of (nBMA-st-MMA-st-DMAEMA) copolymer (PBMD) by RAFT polymerization (st=statistically distributed)

CPAETC (130.7 mg, 4.96×10⁻⁴ mol), nBMA (3.5265 g, 2.48×10⁻² mol), MMA (2.5218 g, 2.52×10⁻² mol), DMAEMA (7.8165 g, 4.97×10⁻² mol), 1,4-dioxane (6.2113 g), a 1.0% by weight ACVA solution in 1,4-dioxane (1.436 g, 14.36 mg ACVA, 5.12×10⁻⁵ mol) and 1,3,5-trioxane (external NMR standard, 23.7 mg) were introduced into a 20 ml microwave vial equipped with a magnetic stirrer. The solution was deoxygenated by bubbling with argon for 10 minutes. The vial was sealed, placed in a 70° C. oil bath and stirred for 21 hours, with samples taken at predetermined times for ¹H—NMR and DMAc-SEC analysis. The polymer was precipitated three times from THF into cold hexane and dried under reduced pressure to give a yellow solid. DMAc-SEC: M_{n,SEC}=25.1 kg mol⁻¹, Ð=1.13.

### EXAMPLE 1B

Synthesis of DMAEMA homopolymer by RAFT polymerization

CPAETC (50.0 mg, 1.9×10-4 mol), DMAEMA (4.54 g, 2.88×10-2 mol), 1,4-dioxane (2.5 g), 1% by weight ACVA in 1,4-dioxane (426 mg, 1.5×10-5 mol) and 1,3,5-trioxane

(external NMR standard, 21 mg) were introduced into a 20 mL microwave vial with magnetic stirrer. The vial was sealed and the solution was deoxygenated by bubbling with argon for approx. 10 min. The vial was placed in an oil bath set at 70° C. and stirred for 7 hours, with samples taken at set times for ¹H—NMR and CHCl3-SEC analysis. The polymer was precipitated three times from THF into cold hexane and dried under reduced pressure to give a yellow solid. CHCl₃-SEC: M_{n,SEC}=14.2 kg mol-1, Ð=1.19.

FIG. 1A shows the structure of the copolymer Eudragit® E100.

FIG. 1B shows the structure of the copolymer represented by RAFT polymerization according to Example 1A.

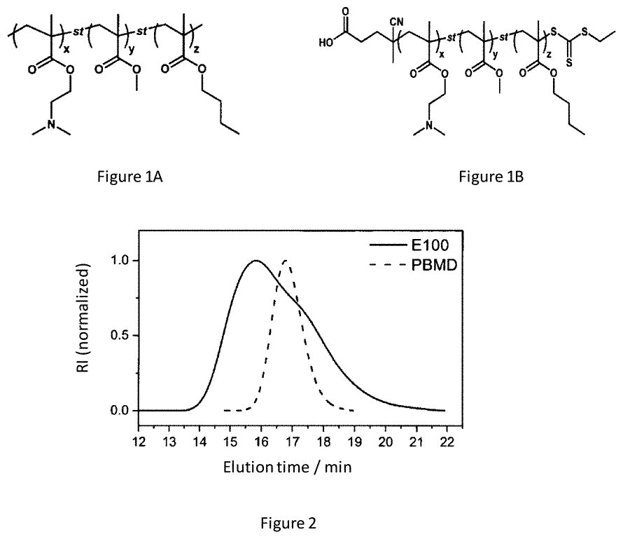

FIG. 2 shows the molecular weight distribution of the copolymer Eudragit® E100 and the copolymer represented by RAFT polymerization according to Example 1A, determined by size exclusion chromatography (SEC).

### EXAMPLE V1

### Formation of nanoparticles and complexation with pDNA (not according to the invention)

For nanoparticle formation, the copolymers produced in Example 1 were dissolved in 2.5 mL acetone (2 mg mL⁻¹). The polymer solution was manually dropped into 5 mL ultrapure water, the resulting nanoparticle suspension was stirred overnight at 800 rpm to remove the organic solvent, and stored at 4° C. until use. To achieve the respective N/P ratio, pDNA was either added in different concentrations during the formation process during the acetone phase and subsequent nanoparticle formation or pDNA was added in different concentrations to the final particle suspension.

### EXAMPLE 2

### Formation of nanoparticulate copolymer-nucleic acid complexes (according to the invention)

Stock solutions of the copolymers produced according to Example 1 were produced by dissolving in 0.2 M acetate buffer (pH 5.8). pDNA, siRNA or mRNA were dissolved in ultrapure water. To produce nucleic acid-polymer complexes, different dilutions of the copolymer as well as the nucleic acid were prepared in HBG buffer (20 mM HEPES, 5% glucose (w/v), pH 7.4) or in 20 mM HEPES buffer to achieve the respective N/P ratios (molar ratio of nitrogen atoms in the copolymer to the phosphate groups in the nucleic acid). After mixing the copolymer solution and the nucleic acid solution, the mixtures were immediately vortexed for 10 seconds. Before use, the resulting copolymer-nucleic acid complexes were incubated at room temperature for at least 15 minutes.

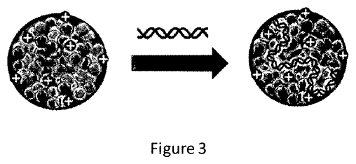

FIG. 3 schematically shows the formation of nucleic acid-copolymer complexes in nanoparticles from the copolymer according to Example 1A (DMAEMA:BMA:MMA=2:1:1).

The use of the cationic and hydrophobic copolymer results in binding, stabilization and protection of the genetic material, formation of stable nanoparticles, stability against competing polyanions and causes endosomal release after uptake by the cell.

### EXAMPLE 3

### General rule for the characterization of nanoparticles and of nanoparticulate copolymer-pDNA complexes

### EXAMPLE 3A

Diameter (z-average) and zeta potential of nanoparticles and pDNA-copolymer complexes were determined by dynamic or electrophoretic light scattering (DLS, Zetasizer Nano ZS, Malvern Instruments, Worcestershire, United Kingdom). For size determination, a refractive index of 1.33 was assumed for ultrapure water and 1.59 for the copolymer. The zeta potential of the nanoparticles produced by precipitation in the presence of organic solvents was determined on the same samples.

### Example 3B

Gel retardation examination

The pDNA binding ability at different N/P ratios was determined by agarose gel electrophoresis. Samples were produced as described for the pDNA-polymer complexes in Examples V1 and 2, ran at 80 V for 1.5 hours on a 1% agarose gel stained with ethidium bromide (EtBr, 0.1 μg mL⁻¹) and imaged using a gel imager (Red™ Imaging System, Alpha Innotech, Kasendorf, Germany).

Example 3C

Ethidium bromide binding assay (EBA) and heparin release assay (HRA)

pDNA complexation and stability of the nanoparticulate copolymer-pDNA complexes were investigated by using an ethidium bromide binding assay and a heparin release assay.

For this purpose, pDNA at a concentration of 15 μg mL⁻¹ was incubated with ethidium bromide for 10 minutes. The polymer stock solutions were diluted in a black 96-well plate (Nunc, Thermo Fisher) to adjust N/P ratios from 1 to 50. Then pDNA was added and the nanoparticulate copolymer-pDNA complexes were incubated at 37° C. for 15 minutes. Ethidium bromide fluorescence intensity was measured at λ_{Ex}=525 nm/λ_{EM}=605 nm. pDNA without copolymer was defined as 100% free DNA. The release of complexed DNA was investigated by gradual addition of heparin and measurement of the resulting changes in ethidium bromide fluorescence intensity. The influence of pH on pDNA binding and pDNA release was investigated by performing the experiment at different pH values in the respective buffers (acetate buffer pH 5 and 5.8 and HBG buffer pH 6.5; 7 and 7.4).

### Example 3D

Transfection of HEK293T cells with EGFP pDNA

The HEK293T cell line was cultured in Dulbecco's modified Eagle's medium (DMEM, 1 g L⁻¹ glucose, 10% (v/v) FBS, 100 g mL⁻¹ penicillin/streptomycin) at 37° C. in a humidified 5% CO₂ atmosphere. For transfection experiments, 0.2*10⁶ cells per mL were seeded into a 24-well plate in 500 μL DMEM supplemented with 10 mM HEPES and left to recover for 24 hours. 1 hour before treatment, the treatment medium was replaced with 450 μL fresh DMEM (10 mM HEPES). Nanoparticulate copolymer-pDNA complexes were freshly prepared as described in Example 2 using egfp pDNA encoding for EGFP or pkmyc pDNA not encoding for a fluorescent protein as negative controls. The cells were treated with 50 μL of a dispersion of nanoparticulate copolymer-pDNA complexes of the indicated N/P ratio and pDNA concentration or with HBG buffer as a control (ctrl) and incubated for 1 or 4 hours. The supernatant was then removed, cultured by ctrl and incubated. The supernatant was then removed, replaced with fresh DMEM (10 mM HEPES) and the cells were further incubated for up to 24 hours. After incubation, cells were separated by trypsin-EDTA, resuspended in HBSS (2% FBS (v/v), 20 mM HEPES) and fluorescence was measured using a flow cytometer (Cytoflex S, Beckmann coulter, Calif., U.S.A.). EGFP expression of viable cells was analyzed by excitation at 488 nm and measurement of emission at 610 nm (bandpass filter 610/20). Fluorescent cells were identified by gating to the negative control.

### Example 3E

Knock-down of GFP in HEK-GFP cells using anti-EGFP siRNA

The HEK-GFP cell line was cultured in Dulbecco's modified Eagle's medium (DMEM, 1 g L⁻¹ glucose, 10% (v/v) FBS, 100 g mL⁻¹ penicillin/streptomycin) at 37° C. in a humidified 5% CO₂ atmosphere. For transfection experiments, 0.1*10⁶ cells per mL were seeded into a 24-well plate in 500 μL DMEM supplemented with 10 mM HEPES and left to recover for 24 hours. 1 hour before treatment, the treatment medium was replaced with 450 μL fresh DMEM (10 mM HEPES). Copolymer-siRNA complexes were freshly prepared as described in Example 2. The cells were treated with 50 μL of the freshly prepared complexes. Complexes with siRNA not directed against GFP and HBG buffer were used as negative controls. 72 hours after treatment with copolymer-siRNA complexes, the supernatant was removed and the cells were separated with trypsin-EDTA. After resuspension in HBSS (2% FBS (v/v), 20 mM HEPES), the cells were analyzed by flow cytometry at an excitation wavelength of 488 nm at 525 nm (bandpass filter 525/40).

### Example 3F

Transfection of HEK293T cells with GFP mRNA

The HEK293T cell line was cultured in Dulbecco's modified Eagle's medium (DMEM, 1 g L⁻¹ glucose, 10% (v/v) FBS, 100 g mL⁻¹ penicillin/streptomycin) at 37° C. in a humidified 5% CO₂ atmosphere. For transfection experiments, 0.2*10⁶ cells per mL were seeded into a 24-well plate in 500 μL DMEM supplemented with 10 mM HEPES and left to recover for 24 hours. 1 hour before treatment, the treatment medium was replaced with 450 μL fresh DMEM (10 mM HEPES). Copolymer-mRNA complexes were freshly prepared as described in Example 2. 50 μL of the copolymer-mRNA complexes were added to the cells and incubated for 4 hours. The supernatant was then removed and the cells were incubated for a further 2 or 20 hours. After incubation, the supernatant was removed, the cells were detached using trypsin-EDTA and resuspended in HBSS (2% FBS (v/v), 20 mM HEPES). Subsequently, the fluorescence of the cells was analyzed by flow cytometry. For this purpose, an excitation wavelength of 488 nm was used and the emission was measured at 525 nm (bandpass filter 525/40).

### Example 3G

PrestoBlue® test to determine viability

For cytotoxicity assays, HEK293T cells were seeded into 24-well plates at a density of 0.2 10⁶ cells per mL (HEK293T) in 500 μL DMEM (10 mM HEPES) and incubated for 24 hours to enable recovery. 50 μL nanoparticulate copolymer-pDNA complexes were added as described in Example 2 to test a concentration range of 0.25-1.5 g mL⁻¹ pDNA.

The cells were incubated with the nanoparticulate copolymer-pDNA complex for 1 or 4 hours. The supernatant was then removed and replaced with 500 μL fresh DMEM (10 M HEPES). 24 hours after treatment, the supernatant was removed and this was replaced with a 10% (v/v) solution of PrestoBlue® (Invitrogen, Calif., U.S.A.) diluted with DMEM medium. The cells were incubated for 45 min and the supernatant was transferred to a 96-well plate (100 μL per well) to determine fluorescence intensity (λ_{Ex} 560 nm, λ_{EM} 590 nm). Cells treated with the HBG buffer were used as control and viability was calculated relative to the buffer control after subtracting the blank (PrestoBlue® without cells).

Investigations of Nanoparticulate Polymer Particles and Nanoparticulate DNA-Polymer Complexes

The binding of genetic material is a crucial step in the gene delivery process, as the genetic material must overcome extracellular and intracellular barriers to reach their target cells. Encapsulation and complexation by cationic polymers, for example, enable protection from nucleases in the bloodstream and the crossing of cell membranes (see in this regard H. Yin, R. L. Kanasty, A. A. Eltoukhy, A. J. Vegas, J. R. Dorkin, D. G. Anderson, Nat Rev Genet 2014, 15, 541-555).

### EXAMPLE 4

### Experiments on the pDNA-binding ability of polymers and the release of pDNA from the complexes formed

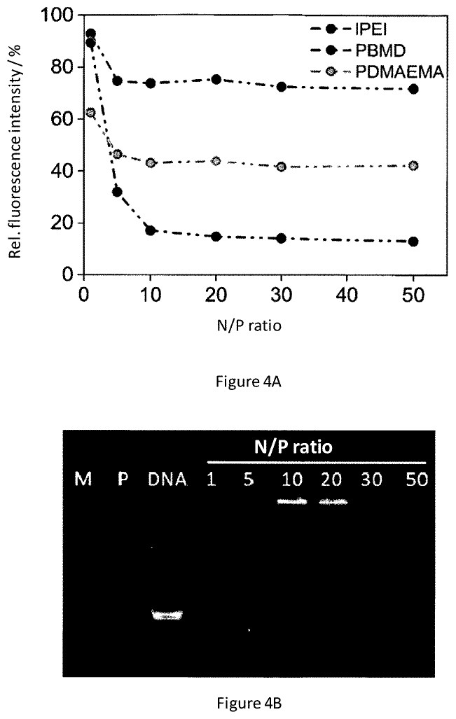

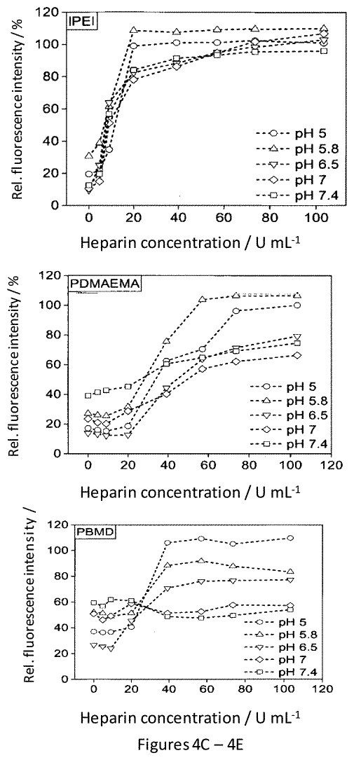

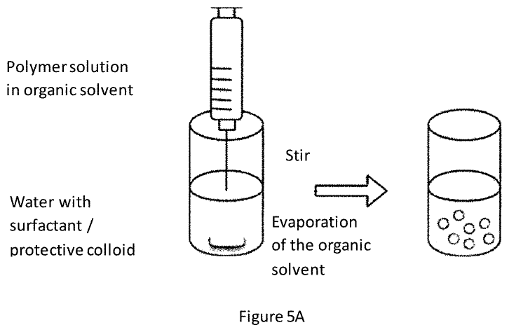

FIG. 4 describes results of experiments on the DNA-binding ability of polymers and the release of pDNA from the complexes formed.

FIG. 4A shows results of an ethidium bromide binding assay (EBA) on DNA-polymer complexes in HBG buffer at pH 7.4. DNA-IPEI complexes, DNA-PDMAEMA complexes and DNA-PBMD complexes were examined.

The binding of the genetic material is crucial for its application in gene delivery. Therefore, the pDNA-binding ability of the polymer was investigated by agarose gel electrophoresis at different N/P ratios. FIG. 4B shows results of agarose gel electrophoresis tests with DNA-PBMD complexes at different N/P ratios.

FIGS. 4C-4E show results of heparin release assays (HRA) for pH-dependent DNA binding and DNA release in the pH range from 5 to 7.4. DNA-IPEI complexes, DNA-PDMAEMA complexes and DNA-PBMD complexes were examined.

The test shown in FIG. 4B confirmed the complete binding of pDNA at N/P ratios above 1. The DNA-polymer interaction can be further characterized by the reversible intercalation of ethidium bromide into the DNA. Strong fluorescence can be observed when this is intercalated into the pDNA. However, this is reduced by the interaction between polymer and genetic material (EBA). The test was carried out at pH 7.4 and N/P ratios from 1 to 50 in order to investigate the effect of an increasing polymer amount on complex formation. In addition to the PBMD polymer, IPEI and a PDMAEMA homopolymer were investigated (FIG. 4A). The latter is composed of the same number of cationic groups as the PBMD polymer in order to evaluate the influence of hydrophobic side chains in the PBMD polymer. It was observed that the relative fluorescence intensity (RFI) decreased with increasing N/P ratios, reaching a plateau at an N/P ratio of 10 (FIG. 4A). IPEI showed the lowest plateau values, followed by PDMAEMA (13% compared to 42%) while PBMD only decreased the RFI to 72%. Since the electrophoresis test showed complete DNA binding above N/P 1, these differences could be due to differences in DNA condensation and thus differently packed complexes, which may lead to different levels of EtBr displacement. Therefore, it can be assumed that the PBMD polymer forms a less dense DNA-copolymer complex compared to IPEI and PDMAEMA.

To investigate the stability of the complexes in the relevant physiological pH range, the dissociation of the complexes was examined at pH values from 5 (endosomal) to 7.4 (blood). Preformed DNA-copolymer complexes with an N/P ratio of 20 were incubated with increasing amounts of heparin as the competing polyanion, and the fluorescence intensity was measured. Dissociation, and thus the release of pDNA from the complex, caused re-intercalation of EtBr. For all polymers, it was observed that ambient pH has an influence on complex formation prior to the addition of heparin. For IPEI (upper FIG. 4C), a notable displacement of EtBr and subsequent release of pDNA was observed at relatively low heparin concentrations (20 U mL⁻¹). pDNA complexes formed at lower pH values required slightly less heparin addition to release the pDNA, but at high heparin concentrations (100 U/ml), pDNA was fully released at all pH values. In general, pDNA release from PDMAEMA complexes required higher amounts of heparin (middle FIG. 4D) and was more strongly influenced by ambient pH in comparison to IPEI. At low pH values (5 and 5.8), 100 U/ml heparin resulted in full release of pDNA, whereas at pH values of 6.5 and above, only 66-79% was released. The PBMD copolymer (lower FIG. 4E) showed an even more pronounced pH-dependent behavior. RFI values before heparin addition range from 27 to 60% and are lower at pH values of 5 to 5.8. The release of the entirety of pDNA was only observed at pH 5 and decreased gradually with increasing pH. At neutral pH, a slight decrease in RFI after heparin addition indicates even stronger complexation of pDNA in the presence of competing polyanions. In general, IPEI was hardly affected by pH, while PDMAEMA and PBMD copolymer showed a weak and the strongest influence of pH, respectively, on the initial RFI values and the subsequent pDNA release.

These results suggest an influence of the pKₐ value and the hydrophobicity of the copolymer on pDNA binding, arrangement in the complex and subsequent pDNA release. For IPEI, a pKₐ value of 8.5 is reported in the literature, while PDMAEMA has a pKₐ of about 7.5. For PBMD, only an apparent pKₐ of 6.8 could be determined, as the polymer precipitated in this pH range during titration.

When calculating the percentage of charges from these pKₐ values, a charge level of 100 to 90% was calculated for IPEI over the entire pH range tested, while PDMAEMA showed a decrease from 100% at pH 5 to 56% at pH 7.4. When calculating the charge level for PBMD with the apparent pKₐ value, the effect is even more pronounced (98% at pH 5 and 20% at pH 7.4). As the charge level and thus the number of cationic groups in the polymer decreases, the ratio of hydrophilic to hydrophobic groups changes, leading to additional hydrophobic interactions and to different arrangements in the complex and thus to altered release behavior of the DNA. This effect is most clearly illustrated in the PBMD copolymer, where the additional hydrophobic side chains of the nBMA and MMA units promote complex stability, especially at neutral pH values, through strong hydrophobic interactions (see in this regard E. J. Adolph, C. E. Nelson, T. A. Werfel, R. Guo, J. M. Davidson, S. A. Guelcher, C. L. Duvall, J. Mater Chem B 2014, 2, 8154-8164). Thus, the PBMD copolymer shows high potential as a gene delivery vector, as high stability and prevention of dissociation at neutral pH in the blood stream is advantageous for systemic application of complexes.

### Example 5

### Experiments on the complexation of pDNA

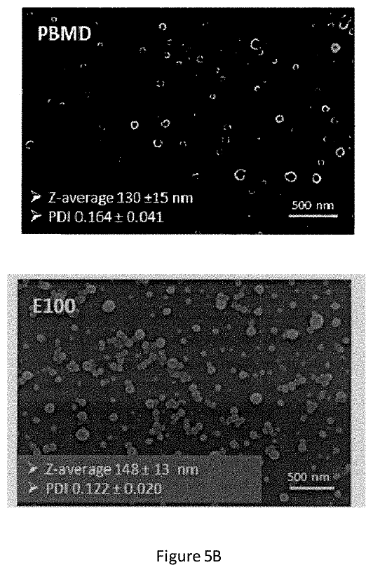

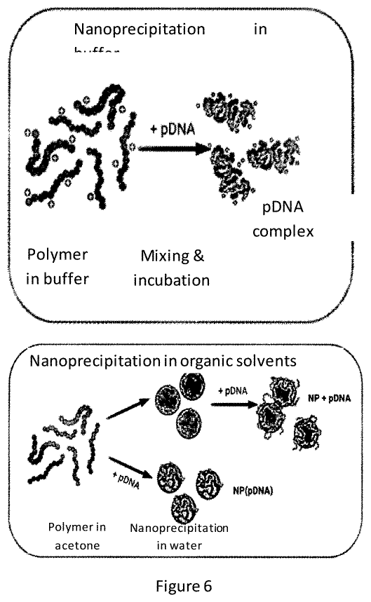

FIGS. 5 and 6 describe results of experiments on the complexation of pDNA in different polymers and the formation of nanoparticles.

FIG. 5A schematically shows the formation of nanoparticles by nanoprecipitation with solvent evaporation technique.

The left side of FIG. 5B shows an electron micrograph (scanning electron microscopy, SEM) of nanoparticles of Eudragit® E100 precipitated from acetone-water mixtures.

The right side of FIG. 5B shows an electron micrograph (scanning electron microscopy, SEM) of nanoparticles of PBMD copolymer precipitated from acetone-water mixtures.

FIG. 6 schematically shows the formation of nanoparticulate DNA complexes of PBMD with pDNA depending on the formulation method used. The left side of FIG. 6 shows the nanoprecipitation with solvent evaporation techniques combined with different techniques of pDNA addition (in-process and post-process addition). The right side of FIG. 6 shows the water-based solvent-free pH-dependent formulation for complexation of pDNA. The obtained nanoparticles were characterized by dynamic light scattering.

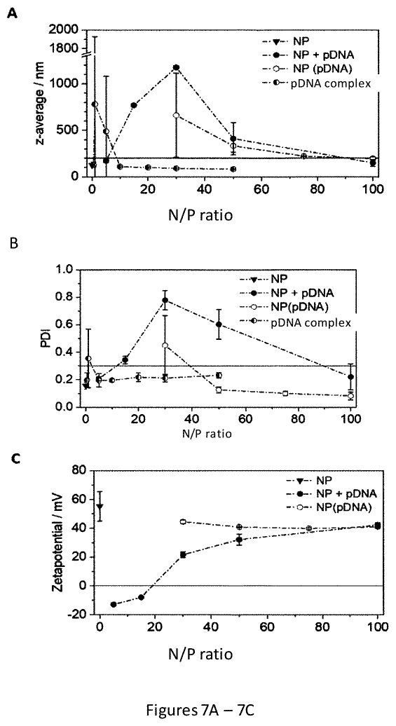

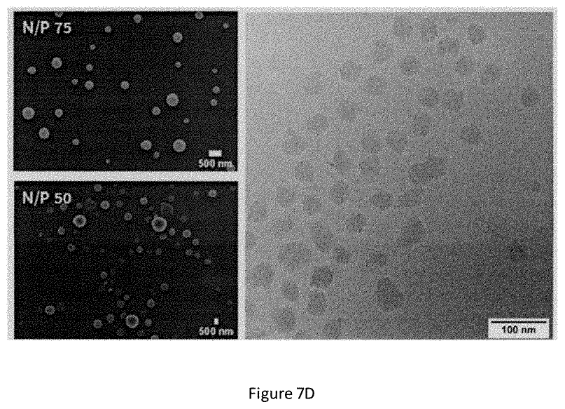

The upper FIG. 7A shows the diameter (z-average) of nanoparticles of PBMD copolymer and pDNA at different N/P ratios produced by precipitation from acetone or aqueous solution. The middle FIG. 7B shows the PDI values of the diameters of nanoparticles of PBMD copolymer and pDNA at different N/P ratios produced by precipitation from acetone or from aqueous solution. The lower FIG. 7C shows the surface charge (zeta potential) of nanoparticles of PBMD copolymer and pDNA produced by means of the solvent evaporation technique from acetone.

The left two FIGS. 7D show electron micrographs (scanning electron microscopy, SEM) of nanoparticles of PBMD copolymer produced at two different N/P ratios by nanoprecipitation from acetone in the presence of pDNA. The right FIG. 7D shows an electron micrograph (cryogenic transmission electron microscopy, cryo-TEM) of nanoparticles of PBMD copolymer produced by nanoprecipitation from aqueous solution.

Since the PBMD copolymer showed high potential for pDNA binding and high complex stability at the pH of blood, the copolymer was used to develop a stable formulation for encapsulating the pDNA. Three different formulation approaches were investigated. The commonly used nanoprecipitation with addition of DNA
a) for the purpose of final nanoparticle suspension or
b) during the formulation process was compared with
c) the complexation of the DNA by the polymer dissolved in the acidic buffer as a pH-dependent water-based nanoprecipitation.

Nanoprecipitation is a widely used method for the formulation of polymeric nanoparticles that allows easy adjustment of nanoparticle size and complexation of a variety of components. Nanoprecipitation of the PBMD copolymer without the addition of pDNA results in particle diameters of about 125 nm (N/P ratio 0) with a positive surface charge (+55 mV) (see FIG. 7C). It was expected that the positive surface charge of the nanoparticles should facilitate the binding and complexation of pDNA to the particle surface. Addition of pDNA to the pre-formulated positively charged nanoparticles (NP+pDNA) generally resulted in increased size and polydispersity of the nanoparticles (FIGS. 7A and 7B). Only at the lowest and highest N/P ratios investigated (N/P 5 and N/P 100) were nanoparticles found with sizes below 200 nm and with acceptable PDI values (PDI<0.250).The surface charge of the particles decreased with increasing DNA amount and shifted to negative values at N/P 10 (FIG. 7C).These results indicate that pre-formulated nanoparticles (NP+pDNA) have limited binding ability for pDNA and do not completely condense the pDNA during complexation. Pre-incubation of the DNA with the polymer dissolved in acetone and subsequent nanoprecipitation in water (NP(pDNA)) leads to a similar N/P ratio-dependent trend of nanoparticle size and homogeneity. Interestingly, the zeta potential remains in the positive range compared to the NP+pDNA formulation. In general, particles obtained by adding the pDNA in the procedure showed a slightly smaller z-average value than those obtained by adding the pDNA afterwards. This could be due to improved interaction of the pDNA with the dissolved polymer chains during the nanoprecipitation process. Overall, this formulation approach did not significantly improve DNA encapsulation, and the amount of complexed pDNA that resulted in stable nanoparticles in the size range below 200 nm with low polydispersity was limited to relatively high N/P ratios (N/P 100).

The copolymer Eudragit® E100 was also used for nanoparticle formation and pDNA complexation, in addition to the polymers PDMAEMA, IPEI and PBMD copolymer described above.

Formulation methods based on polymers and organic solvents for nanoprecipitation and related formulation methods are known in principle (see R. Jain, P. Dandekar, B. Loretz, M. Koch, C.-M. Lehr, MedChemComm 2015, 6, 691-701; N. Kanthamneni, B. Yung, R.J. Lee, Anticancer research 2016, 36, 81-85; M. Gargouri, A. Sapin, S. Bouali, P. Becuwe, J. Merlin, P. Maincent, Technology in cancer research & treatment 2009, 8, 433-443).

According to the invention, a new organic solvent-free, water-based formulation method is provided, which was inspired by the joint complexation of DNA with water-soluble polymers. The Eudragit® E100 copolymer used here and the PBMD copolymer produced by RAFT are soluble under acidic conditions due to the protonation of the DMAEMA groups.

To implement the method according to the invention, sodium acetate buffer (pH 5.8) was used to dissolve the copolymers before mixing with pDNA. This formulation approach resulted in nanoparticles in the range of 100 nm with decreasing diameter at higher N/P ratios and with PDI values around 0.25 for all N/P ratios tested (see FIGS. 7A and 7B). Overall, much lower N/P ratios were obtained with this formulation method, indicating better complexation and condensation of the pDNA by the PBMD copolymer. This could be due to a higher charge fraction of the polymer when dissolved in the acetate buffer, and thus better complexation and condensation of the pDNA. Low N/P ratios are preferred for gene delivery. At lower N/P ratios, lower amounts of copolymer can be used, resulting in more efficient gene delivery and lower toxic potential due to reduced copolymer concentrations. Since the complex formulation meets these criteria, this formulation was tested for in vitro transfection compared to the NP+pDNA formulation. The NP(pDNA) formulation was not further investigated as the low N/P ratios tested in the in vitro experiments (10 and 20) could not be produced due to failure and strong aggregation during the formulation process.

### Example 6

Experiments on pDNA transfection efficiency in HEK293T cells

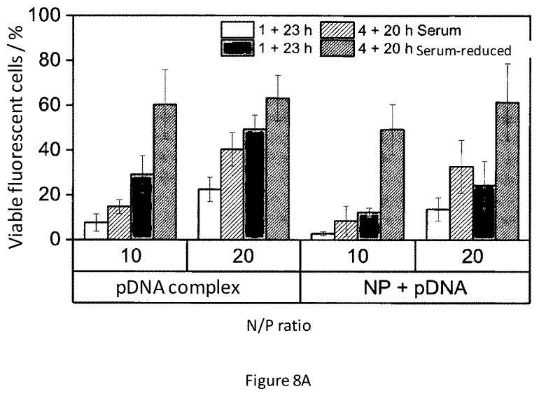

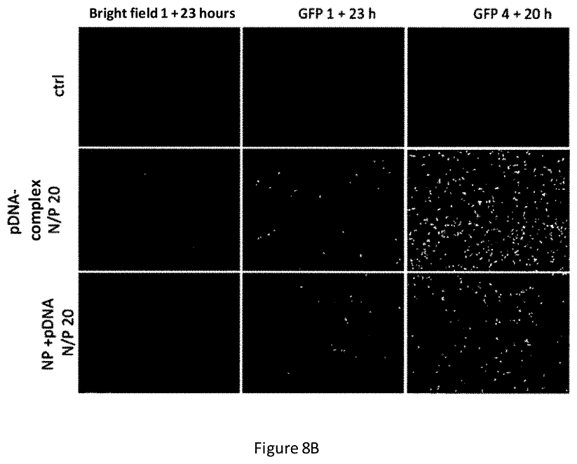

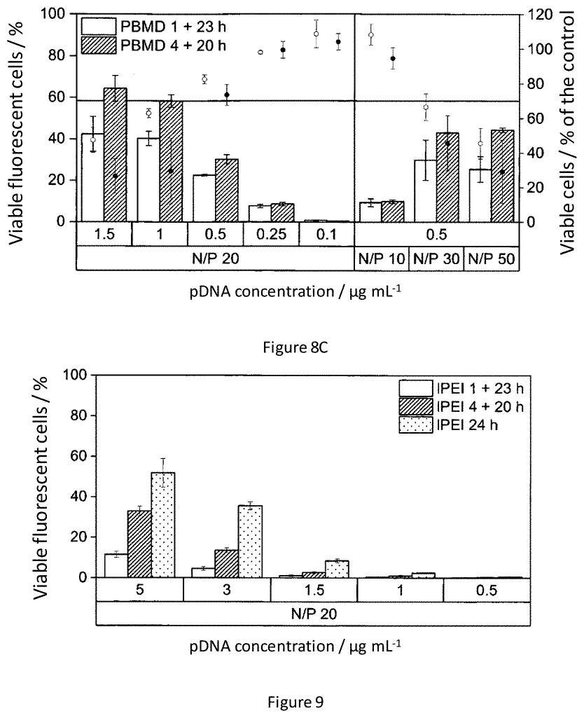

FIGS. 8, 9 and 10 describe results of experiments on the transfection efficiency of pDNA-copolymer complexes in cells.

FIG. 8A shows comparisons of formulation methods and their transfection efficiency measured by flow cytometry.

FIG. 8B describes results obtained by fluorescence microscopy.

FIG. 8C describes the results of the optimization of the N/P ratio and the pDNA concentration in complexes with PBMD copolymer.

FIG. 9 describes results of transfection experiments with complexes of pDNA with IPEI.

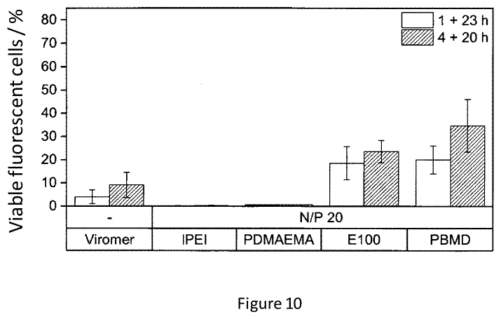

FIG. 10 describes results of transfection experiments with complexes of pDNA with PBMD copolymer as well as with various commercially available polymers such as Eudragit® 100.

The transfection efficiency of the pH-dependent formulation prepared by pDNA addition after nanoparticle generation was investigated by transferring EGFP-encoding pDNA into HEK293T cells and measuring the EGFP expression after 24 hours. The transfection efficiency of the formulation was determined by the addition of pDNA after nanoparticle generation. Two different N/P ratios were tested in the non-toxic region (FIG. 8A) of the copolymer. Complexes were used which had been generated by precipitation of the nanoparticles from acetone followed by pDNA addition (right part of FIG. 8A and C) and which had been generated by precipitation of the nanoparticles from acidic aqueous solution in the presence of pDNA (left part of FIG. 8A). Two different media containing different levels of FCS were also used. One medium contained 10% FCS and another medium was serum-reduced (Opti-MEM, 2% FCS). The left two columns in each part of FIG. 8A describe the results in the serum-containing medium; the right two columns in each part of FIG. 8A describe the results in serum-free medium.

FIG. 8B shows fluorescence microscopy examinations of the transfected cells after 1+23 incubation time (without sample) or after 4+20 hours incubation time (sample). Complexes were used which had been generated by precipitation of the nanoparticles from acetone followed by addition of pDNA (bottom row of FIG. 8B) and which had been generated by precipitation of the nanoparticles from acidic aqueous solution in the presence of pDNA (middle row of FIG. 8B). N/P ratios of 20 were used in each case.

In addition, the top row of FIG. 8B presents results showing cells that received HBG buffer instead of nanoparticles or complexes, serving as an internal control within the experiment (ctrl).

In general, both formulations, those produced by precipitation from acetone and those produced by precipitation from acidic aqueous solution, showed transfection in HEK293T cells with higher efficiency under serum-reduced conditions and at higher N/P ratios. The pH-dependent formulation showed higher and more consistent transfection efficiencies compared to the formulation produced by precipitation from acetone. This could be due to aggregates formed by the formulation of pDNA addition after the procedure with particle diameters>4 500 nm and with higher polydispersity in the DLS measurements (FIGS. 7A and 7B). Therefore, the sedimentation of the different species in the formulation differs depending on their size, leading to variations in cellular uptake and transfection efficiency.

The pH-dependent formulation leads to nanoparticles with less variation in sizes without the formation of aggregates. This could lead to more controlled uptake into cells and more consistent transfection rates.

Since the pH-dependent formulation yields particles with preferred N/P ratio, size and homogeneity, this formulation was further investigated and optimized for transfection efficiency. The conditions during transfection with PBMD-copolymer complexes were optimized for high transfection efficiency while maintaining high cell viability. Furthermore, the minimum amount of copolymer and pDNA was to be identified. Different pDNA concentrations were investigated, keeping the N/P ratio constant at 20, as this was found to be optimal (see left half of FIG. 8C). Subsequently, the ideal pDNA concentration (0.5 mg mL⁻¹) was further optimized by varying the N/P ratio (see right half of FIG. 8C).

The results from FIG. 8C were compared with complexes formed with p-DNA and IPEI as the gold standard (see FIG. 9). Overall, complexes formed with PBMD-copolymer showed high transfection efficiencies at lower pDNA concentrations and shorter incubation times compared to complexes formed with pDNA and IPEI.

Incubation times of 4 hours resulted in higher transfection efficiencies for complexes of pDNA and PBMD copolymer compared to complexes of p-DNA and IPEI over the range of concentrations and N/P ratios investigated, but also resulted in reduced cell viability at higher polymer concentration (=larger N/P ratio) (see FIGS. 8C and 9). When the pDNA concentration was increased (8.59% at 0.25 μg mL⁻¹ to 64.3% at 1.5 μg mL⁻¹, 4 hours incubation time), an increase in transfection efficiency was observed (see left part of FIG. 8C). Increasing the N/P ratio at constant pDNA concentration resulted in higher efficiencies (see right part of FIG. 8C), but led to increased cell detachment and cell death. Therefore, 0.5 μg mL⁻¹ at an N/P ratio of 20 was chosen as the ideal conditions for effective pDNA transfection with the PBMD-copolymer complex.

Compared to the gold standard IPEI, the pDNA concentration could be reduced to 0.5 μg mL⁻¹, which still resulted in 22.5% fluorescent cells after 1 hour incubation, and which was not possible with IPEI in this short incubation time and in this investigated pDNA concentration range (0.5 to 5 μg mL⁻¹) (see left part of FIG. 8C with FIG. 9). To achieve higher transfection efficiencies, either the pDNA concentration must be increased or incubation times of up to 24 hours are required (see FIG. 9). PDMAEMA showed almost no transfection even with very high pDNA concentrations of 5 μg mL⁻¹ and incubation times of 24 hours. These results support the hypothesis of the influence of hydrophobic side chains on the in vitro performance of cationic polymers as gene delivery vectors.

FIG. 10 shows the transfection efficiency of pDNA complexes with PBMD copolymer, with PDMAEMA homopolymer and with the commercial polymers Eudragit® E100, Viromer red and IPEI.

The formulations for PBMD and EUDRAGIT® E100 were produced by precipitation from acidic aqueous solution in the presence of pDNA. Expression of eGFP in HEK293T cells after transfection was measured by flow cytometry. HEK293T cells were transfected at an N/P ratio of 20 and a pDNA concentration of 0.5 μg mL⁻¹.

The pDNA-PBDM complex formulation technique was further applied to the commercial polymer Eudragit® E100 and investigated under the optimized transfection conditions. In addition, the transfection efficiency was compared with the commercial transfection agent Viromer Red. Overall, the pH-dependent formulation was applicable to the commercial polymer EUDRAGIT® E100, which showed comparable transfection efficiencies after 1 hour incubation with the pDNA-PBMD-copolymer complex, but showed a slightly lower efficiency after 4 hours incubation. This could be due to the slightly higher DMAEMA content in the PBMD copolymer. A higher cationic content would lead to higher DNA binding and increased membrane activity.

Both copolymers are clearly superior in performance to the commercial transfection agents Viromer Red and IPEI.

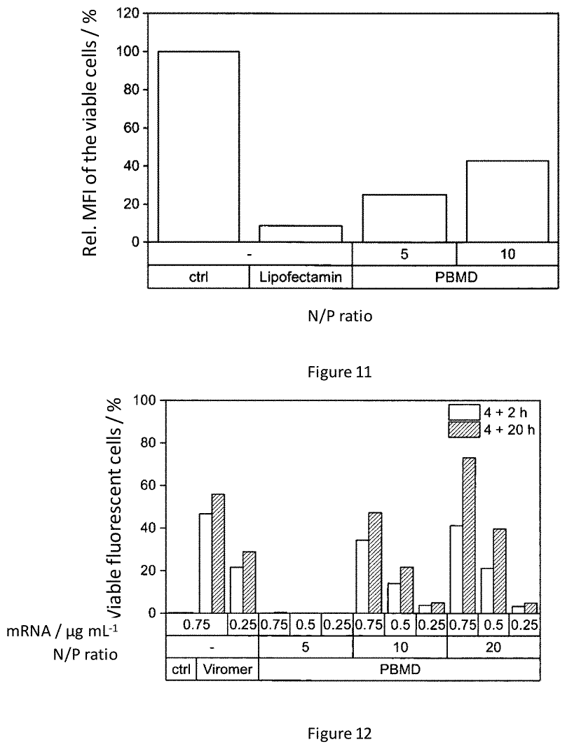

FIG. 11 describes results of experiments on the transfection efficiency of siRNA complexes in cells.

To determine knock-down efficiency, anti-GFP siRNA was introduced into HEK-GFP cells showing stable expression of GFP by copolymer-siRNA complexes produced by precipitation from aqueous acidic solution. Knock-down efficiency after 72 hours incubation was compared with the gold standard Lipofectamine. Cells treated only with HBG buffer served as control. Both Lipofectamine and copolymer-siRNA complexes showed a reduction in the number of fluorescent cells (GFP positive cells) compared to the control (100%). Copolymer-siRNA complexes showed a reduction of GFP positive cells to 42.9% at an N/P ratio of 10 and a further reduction to 25.2% at an N/P ratio of 5. Lipofectamine resulted in a reduction to 8.6%. Thus, the PBMD copolymer also shows a high potential for the introduction of siRNA into cells, which can still be improved by further optimization of the transfection conditions such as incubation time, N/P ratio and siRNA quantity.

FIG. 12 describes results of experiments on the transfection efficiency of mRNA complexes in cells.

The precipitation of complexes of genetic material and copolymer was also applied to the production of copolymer-mRNA complexes. The transfection efficiency of these complexes was determined by introducing GFP mRNA into HEK293T cells. Different N/P ratios (5, 10, 20) and mRNA concentrations (0.25 to 0.75 μg mL⁻¹) were tested. The cells were incubated for 4 hours with the complexes. 6 and 24 h after the start of treatment, GFP expression was determined by fluorescence measurement in flow cytometry. The transfection efficiency of the copolymer-mRNA complexes was compared with the commercially available Viromer Red, which was developed for the transfection of mRNA and shows high efficiencies in the literature. Cells incubated only with HBG buffer or mRNA without copolymer served as negative controls. Both Viromer Red and copolymer-mRNA complexes led to mRNA concentration-dependent GFP expression in HEK293T cells. A higher mRNA concentration entailed higher expression levels, while an increase in the N/P ratio in the copolymer-mRNA complexes also entailed an increase. Viromer Red showed the highest transfection efficiency at an mRNA concentration of 0.75 μg mL⁻¹and an incubation time of 24 hours (55.8%). The highest transfection efficiency was achieved after 24 hours incubation with copolymer-mRNA complexes with an N/P ratio of 20 and an mRNA concentration of 0.75 μg mL⁻¹ (73.1%). In addition to introducing pDNA and siRNA into cells, the PBMD copolymer thus also shows a high potential for introducing mRNA into cells.

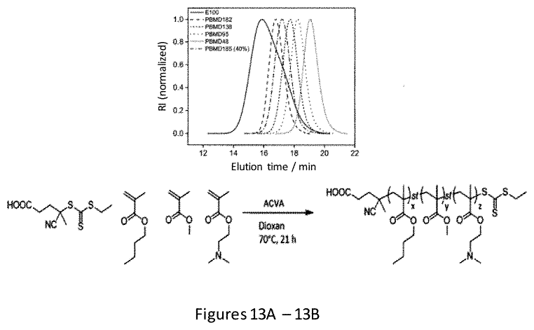

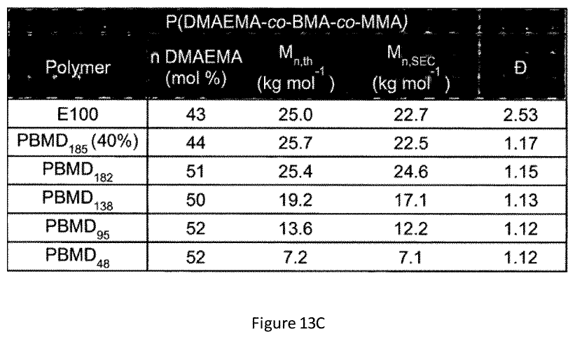

FIGS. 13A-13C describe results for the synthesis and characterization of different PBMD copolymers with varying molecular weights.

They show the synthesis route for the production of the PBMD copolymers (described in Example 1A, FIG. 13A) and the molecular weight distribution and polydispersity of the copolymers prepared by RAFT polymerization analogous to Example 1A, as determined by size exclusion chromatography (SEC) (FIGS. 13B and 13C).

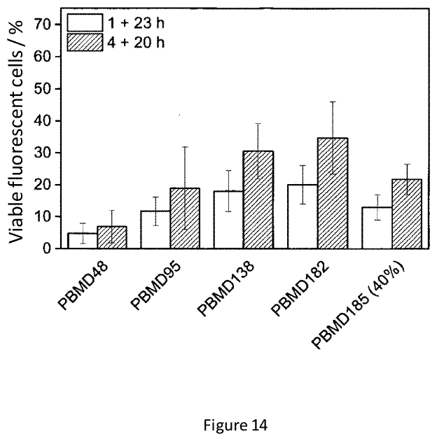

FIG. 14 shows the transfection efficiency of pDNA complexes with PBMD copolymers of different molecular weight in HEK293T cells.

The transfection efficiency of the PBMD polymer library was investigated in HEK293T cells under the optimized conditions of PBMD182 copolymer. egfp-pDNA was complexed by the copolymers dissolved in acetate buffer and incubated with the cells for either 1 or 4 hours. Overall, all copolymer-pDNA complexes within the PBMD library showed transfection efficiency in HEK293T cells. The results show a clear influence of molar mass and molar content of DMAEMA on transfection efficiency. Copolymer-pDNA complexes with a molecular weight of >15 kDa and a molar DMAEMA content of 50% show transfection efficiencies of 18% and higher depending on the incubation time. The highest transfection efficiencies were achieved with the PBMD182 copolymer (the copolymer prepared according to Example 1A) after 4 hours incubation with the copolymer-pDNA complexes (34.6%). The PBMD185(40%) copolymer with a molar content of DMAEMA of 40% and with a molar mass >15 kDa showed comparatively slightly lower transfection efficiencies (13% after 1 hour incubation, 21.7% after 4 hours incubation) compared to polymers with 50% molar DMAEMA content. The formation of copolymer-pDNA complexes can thus also be transferred to polymers of different molar mass.

1. Nanoparticles comprising complexes formed from nucleic acids and cationic copolymers containing the recurring structural units of the formulae (Ia) and (Ib) 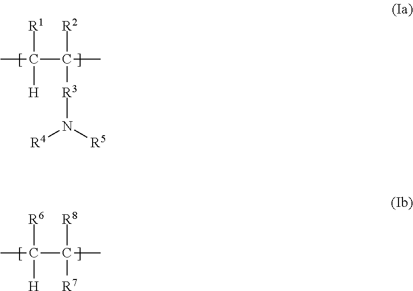
wherein
R¹ and R⁶ are, independent of each other, hydrogen, alkyl or —COOR⁹,
R² and R⁷ are, independent of each other, hydrogen or alkyl,
R³ is selected from the group consisting of 613 O—R¹⁰—, —COO—R¹⁰—, —CONH—R¹⁰— or —R¹⁰—,
R⁴ is hydrogen, alkyl, cycloalkyl, aryl, aralkyl or alkylaryl,
R⁵ is hydrogen, alkyl, cycloalkyl, aryl, aralkyl, alkylaryl or —(alkylene-NH—)ₘ-alkyl, or
R⁴ and R⁵ form a heterocyclic ring together with the nitrogen atom they have in common,
R⁸ is selected from the group consisting of —O—R¹¹, —COO—R¹¹, —CONH—R¹¹ or —R¹¹,
R⁹ and R¹¹ are, independent of each other, hydrogen or a monovalent organic radical,
R¹⁰ represents a bivalent organic radical, and
m is an integer from 1 to 5, with the requirement that the nanoparticles have a diameter (z-average) of less than or equal to 900 nm, determined by dynamic light scattering, and that the molar ratio of nitrogen atoms in the copolymer to phosphate groups in the nucleic acid is between 1 and 200.

2.-7. (canceled)

8. The nanoparticles according to claim 1, characterized in that the copolymers comprise a recurring structural unit of the formula (Ia) and two different recurring structural units of the formula (Ib), in which R¹ and R⁶ are hydrogen, R² and R⁷, independent of each other, are hydrogen or methyl, in particular methyl, R³ is —COO—R¹⁰—, R¹⁰is ethylene, R⁴ and R⁵, independent of each other, are C₁-C₆ alkyl, in particular methyl, and R⁸ is —COO—R¹¹, where, in one recurring structural unit of the formula (Ib), R¹¹ is C₁-C₃ alkyl, in particular methyl, and, in another recurring structural unit of the formula (Ib), R¹¹ is C₄-C₆ alkyl, in particular n-butyl.

9. The nanoparticles according to claim 1, characterized in that, in addition to the recurring structural units of the formulae (Ia) and (Ib), the copolymers also contain further recurring structural units of the formula (Ic) 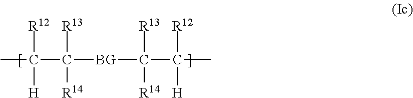
wherein
R¹², R¹³ and R¹⁴, independent of each other, are hydrogen or alkyl, preferably hydrogen or C₁-C₆ alkyl, particularly preferably hydrogen or methyl, and BG represents a bivalent organic bridging group with ether, ester, amide, sulfide, phosphate or disulfide groups.

10. The nanoparticles according to claim 1, characterized in that the molar proportion of the recurring structural units of the formula (Ia) is between 10 and 75%, preferably between 15 and 65% and very particularly preferably between 20 and 55%, based on the total cationic copolymer, and in that the molar proportion of the recurring structural units of the formula (Ib) is between 90 and 25%, preferably between 85 and 45% and very particularly preferably between 80 and 45%, based on the total cationic copolymer.

11. The nanoparticles according to claim 1, characterized in that their particle diameters (z-average) range between 40 and 250 nm, determined by light scattering.

12. The nanoparticles according to claim 1, characterized in that their polydispersity index of particle size distribution, measured with the Malvern Zetasizer Nano ZS (Malvern Instruments, Worcestershire, United Kingdom) using cumulant analysis of the correlation function (ISO13321, ISO22412), ranges between 0.05 and 0.4, preferably between 0.1 and 0.4 and particularly preferably between 0.1 and 0.3.

13. The nanoparticles according to claim 1, characterized in that the polydispersity index of molar mass distribution of the cationic copolymers used ranges between 1.0 and 3.0, preferably between 1.01 and 2.6.

14. The nanoparticles according to claim 1, characterized in that they have a transfection efficiency for pDNA of 15 to 50% (viable fluorescent cells), in particular of 20 to 45%, after 1 hour incubation time of cells with the nanoparticles and 23 hours subsequent incubation of the cells in growth medium without nanoparticles.

15. The nanoparticles according to claim 1, characterized in that the molar ratio of nitrogen atoms in the copolymer to phosphate groups in the nucleic acid is between 1 and 100, preferably between 2.5 and 100 and very particularly preferably between 5 and 50.

16. The nanoparticles according to claim 15, characterized in that they have diameters between 40 and 250 nm, determined by DLS, and a polydispersity index of particle diameters between 0.1 and 0.3.

17. The nanoparticles according to claim 16, characterized in that their molar ratio of nitrogen atoms in the copolymer to phosphate groups in the nucleic acid is between 10 and 30.

18. The nanoparticles according to claim 1, characterized in that they are present dispersed in water, and in that their proportion by weight in the dispersion is between 0.01 and 20%, preferably between 0.05 and 5%.

19. A method for the production of nanoparticles comprising the following measures:
i) production of an aqueous solution of a cationic copolymer containing the recurring structural units of the formulae (Ia) and (Ib) according to claim 1 having a pH between 3 and 6.5,
ii) production of an aqueous solution of a nucleic acid,
iii) mixing of the two solutions produced in steps i) and ii) in a selected quantity ratio of nucleic acid and copolymer to give a desired molar N/P ratio of nitrogen atoms in the copolymer to phosphate groups in the nucleic acid between 1 and 200, and
iv) agitation of the resulting mixture.

20. The method according to claim 19, characterized in that it comprises, as step v), an incubation of the obtained mixture.

21. The method according to claim 19, characterized in that the aqueous solution of the cationic copolymer for step i) contains a buffer, in particular an acetate buffer, citrate buffer, lactate buffer, phosphate buffer, phosphate-citrate buffer or mixtures of the buffers.

22. The method according to claim 19, characterized in that the aqueous solution of the nucleic acid for step ii) has a pH from 6.5 to 8.5, in particular from 6.8 to 7.5.

23. The method according to claim 22, characterized in that the aqueous solution of the nucleic acid for step ii) contains a buffer, in particular an HBG, HEPES, BIS-TRIS propane or TRIS buffer.

24. A method for gene delivery into cells comprising the following steps:
A) bringing cells into contact with an aqueous suspension comprising the nanoparticles according to claim 1, and
B) subsequent incubation.

25. The method according to claim 24, comprising the following steps:
C) provision of a cell culture in a bioreactor or incubator,
D) addition of an aqueous suspension comprising the nanoparticles according to at least one of claims 1 to 18,
E) distribution of the aqueous suspension in the cell culture, and
F) subsequent incubation.

26. The method according to claim 24, characterized in that the cells used are selected from the group consisting of single cells, tissues or cell cultures.

27. (canceled)
